(19) United States
(12) Patent Application Publication
(10) Pub. No.: US 2010/0145167 A1
(43) Pub. Date: Jun. 10, 2010

(54) PILLOW HAVING APPARATUS FOR DETERMINING SLEEPING STATE UNDER UNRESTRICTED NON-SELF-AWARENESS CONDITION

(75) Inventor: Jae Joong IM, Jeonju (KR)

Correspondence Address: LRK Patent Law Firm, 1952 Gallows Rd, Suite 200, Vienna, VA, 22182, US

(73) Assignee: HANBYUL MEDITECH CO., LTD., Jeonju (KR)

(21) Appl. No.: 12/702,778

(22) Filed: Feb. 9, 2010

(30) Foreign Application Priority Data
Aug. 8, 2008 (KR) 10-2008-0077751

(51) Int. Cl. A61B 5/00; A47C 20/00

(52) U.S. Cl. 600/301

(57) ABSTRACT

Provided is a pillow having an apparatus for determining a sleeping state under an unrestricted non-self-awareness condition. The apparatus includes a sensor for detecting analog bio-information on a pulse, breathing, body movement, snore. bruxism for a user putting his/her head on the pillow, a signal processing unit for filtering off and amplifying the analog bio-information detected by the sensor, and a control unit for converting the analog bio-information output from the signal processing unit into a digital signal, storing the digital signal therein, extracting parameters from the detected bio-information to provide a sleeping status as a quantitative indicator. The sensor is installed in the pillow. The sensor is inserted into a pad type housing so that the sensor does not directly contact an interior material of the pillow. The housing is provided on an upper portion of the pillow so that minute vibration and movement of the user's head, which are incurred by the user's breathing, pulse, body movement, and snore, is effectively transferred to the sensor.

### BACKGROUND OF THE INVENTION

The present invention disclosed herein relates to an apparatus for detecting bio-information in a sleeping status, and more particularly, to a pillow with an apparatus for inference of the sleeping status, which can detect and analyze a pulse, breathing, and body movement using a sensor detecting variation of vibration or pressure in unrestriction and non-self-awareness states and particularly can provide a quantitative indicator determining the sleeping status.

When a person does not sleep soundly, he/she lacks the ability to concentrate and has a very low level of accomplishments and thus falls asleep at the wheel, which causes traffic accidents. Since the quality deterioration of the sleep causes a body disease and causes the traffic accidents, there is a need for a comfortable sleeping environment.

Accordingly, a variety of methods for measuring and determining physical and psychological variations in sleep have been required to provide the most comfortable sleeping environment or seek a sound sleeping method. For example, a method for inferring the sleeping status from waveforms attained by testing the electrocardiogram, brainwave, breathing, electromyogram, eye movement, body movement, and the like is well known. Particularly, the snore is also classified as a habit disease which disturbs the sound sleep. According to an epidemiological survey, the snore is likely to be developed as the sleep-apnea. In addition, the snore may cause the cardiac/vascular, brain disease, high blood pressure, and diabetes. As the percentage of the obese population is increasing, the number of the snore patients is also increasing.

In a typical method for determining the sleeping status, a plurality of sensors for detecting above-stated bio-signals are directly attached on a skin of sleeper and the sleeper are in a restricted status by wires connecting the sensors to a measuring apparatus. In addition, since bio-information of many channels is required, a reliable, simple method for classifying sleeping steps is required to economically analyzing the sleeping status. Although a couple of researches have been making much progress to overcome the above-described limitations, there are yet limitations to be overcome.

Korean Pat. No. 10-2003-0083422 and Japanese Laid-open Pat. No. P2004-173725A disclose heartbeat/breathing measuring apparatuses that output a heartbeat cycle, a heartbeat rate, a breathing cycle, and a breathing rate from heartbeat and breathing components by detecting an oscillating frequency of an oscillating circuit. Theses apparatuses are configured to measure the heartbeat and breathing rates without restricting the human body. However, there is a limitation in that the testee should be always at the sensor location. In addition, Since an LC oscillating circuit is used to measure the bio-signal, the testee may be affected by electromagnetic waves. Japanese Pat. No. P2004-89267A discloses an algorithm for measuring a sleeping depth using the bio-signal measured by the above-described method. According to this algorithm, the bio-signals on the heartbeat and breathing are detected by a sensor and only the sleeping steps are analyzed and provided by inferring the sleeping depth using the heartbeat, breathing, and body movement. That is, this algorithm does not provide other information on the sleeping status except for the sleeping steps.

Korean Pat. No. 10-2004-0013374 discloses a method and apparatus for simultaneously measuring an electrocardiogram and breathing without self-awareness. This apparatus is designed to simultaneously measure the electrocardiogram and breathing of the testee lying in a bed without the self-awareness. In addition, Japanese Laid-open Pat. No. P2002-224051 A discloses an apparatus for monitoring an abnormal state using a body temperature, heartbeat rate, breathing rate, and body movement that are attained by measuring bio-signals of an underweight baby or infants. This apparatus has a limitation in that conductive fabric patch should be always in contact with the testee. That is, this apparatus is not a non-contact measuring type apparatus. In addition, since the movement of the testee who is sleeping is restricted, there is a limitation to the substantial application of this apparatus. Japanese Laid-open Publication No. P2005-205023A discloses an apparatus for measuring the heartbeat and breathing rates of a user. This apparatus includes a tube filled with water is disposed between plates and is installed in a pillow. The heartbeat and breathing rates are measured by measuring pressure variation of the water using a pressure sensor. However, this apparatus is not proper for the body movement or snore.

Japanese Laid-open Publication No. 2005-279113 discloses a wrist watch type sleeping status determining apparatus. This apparatus infers the sleeping status using a pressure sensor that is to be attached on a finger and an acceleration sensor that is to be attached on a palm. The pressure sensor detects a pulse interval. This apparatus provides information on the frequencies of the body movement, sleep-apnea, and unequal pulse by estimating an autonomic nerve indicator and a body movement state. However, since the sensors should be attached on the body of the user who is sleeping, this apparatus is not the unrestricting-unawareness type apparatus. A system for measuring movement of a patient using capacitance type sensors is also well known. This system includes a mat having 64 measuring points. A pressed distribution by the weight of the patient is attained from a capacitance distribution including information a three-dimensional location and pressure. Using the pressed distribution, the variation of the body location and position is inferred. However, this system does not provide information of the sleep.

As described above, the sleeping status closely relates to the body movement, snore, heartbeat, and breathing. Therefore, there is a need to accurately detect variation of bio-information in a free sleeping status without restriction and self-awareness. To achieve this, the sensors should be properly arranged and applied. In addition, the quantitative indicator of the sleeping status should be provided by the detected bio-information.

### SUMMARY OF THE INVENTION

The present invention provides a pillow having an apparatus for determining a sleeping state under an unrestricted non-self-awareness condition, which can quantitatively infer a sleeping status by detecting a pulse, breathing, body movement, snore, bruxism, and the like using a sensor detecting variation of vibration or pressure in unrestriction and non-self-awareness states and particularly can provide a quantitative indicator determining the sleeping status.

Embodiments of the present invention provide pillows having an apparatus for determining a sleeping state under an unrestricted non-self-awareness condition, the apparatus including: a sensor for detecting analog bio-information on a pulse, breathing, body movement, snore, bruxism for a user putting his/her head on the pillow; a signal processing unit for filtering off and amplifying the analog bio-information detected by the sensor; and a control unit for converting the analog bio-information output from the signal processing unit into a digital signal, storing the digital signal therein, extracting parameters from the detected bio-information to provide a sleeping status as a quantitative indicator, wherein the sensor is installed in the pillow; the sensor is inserted into a pad type housing so that the sensor does not directly contact an interior material of the pillow; and the housing is provided on an upper portion of the pillow so that minute vibration and movement of the user's head, which are incurred by the user's breathing, pulse, body movement, and snore, is effectively transferred to the sensor.

In some embodiments, the sensor may be fixed on a central portion of the pad type housing and selected from the group consisting of a piezoelectric ceramic, a piezoelectric film, a piezoelectric cable, a resistive pressure sensor, a load cell, and an acceleration sensor.

In other embodiments, the housing may include upper and lower pads for protecting the sensor and a fixing support for fixing the upper and lower pads. At this point, the housing may be formed of aluminum or plastic so that minute vibration and pressure variation signals during the user's sleep is efficiently transferred to the sensor and the sensor is fixed at the central portion of the housing.

In still other embodiments, an elastic member may be provided between the sensor and the upper pad so that minute vibration and pressure variations are efficiently transferred from the upper pad to the sensor, wherein the elastic member is formed of rubber.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIGS. 1A to 1C are views illustrating a use state of a pillow having an apparatus for determining a sleeping status according to an exemplary embodiment;

FIGS. 2A to 2C are respectively exploded perspective, front cross-sectional, and top cross-sectional views of a sensor unit of the sleeping status determining apparatus of FIGS. 1A to 1C;

FIG. 3 is a block diagram a sleeping status determining apparatus according to one exemplary embodiment;

FIG. 4 is a flowchart illustrating operation of a control unit of a sleeping status determining apparatus according to one exemplary embodiment; and

FIG. 5 is a flowchart illustrating a process for deducing parameters of a sleeping status determining apparatus according to one exemplary embodiment.

### DETAILED DESCRIPTION OF PREFERRED EMBODIMENTS

Preferred embodiments of the present invention will be described below in more detail with reference to the accompanying drawings. The present invention may, however, be embodied in different forms and should not be constructed as limited to the embodiments set forth herein. Rather, these embodiments are provided so that this disclosure will be thorough and complete, and will fully convey the scope of the present invention to those skilled in the art.

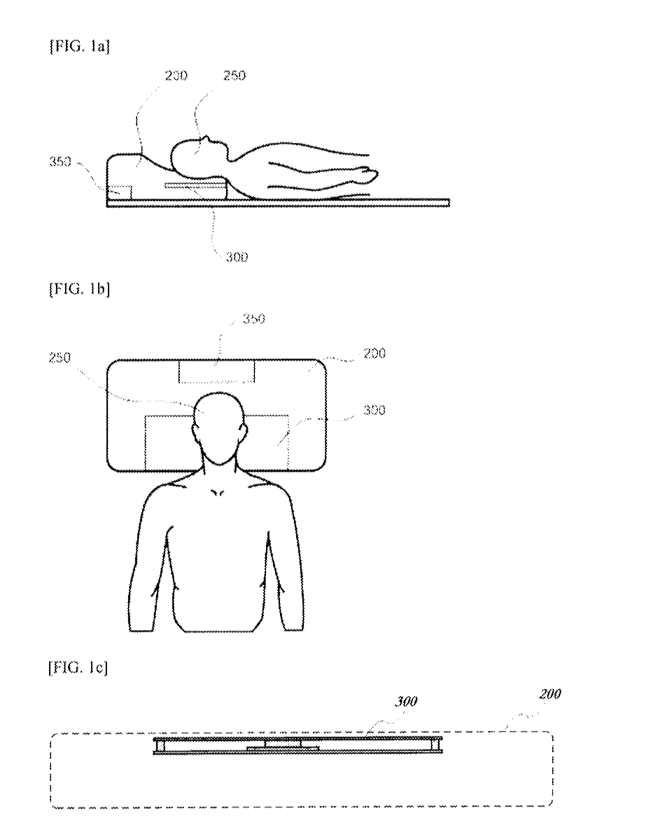

FIG. 1A to 1C are schematic views illustrating an apparatus for determining an unrestricted sleeping status according to an embodiment. An apparatus includes a sensor unit that is disposed in a pillow 200 to detect variations of vibration and pressure incurred by a pulse, breathing, body movement, snore, bruxism of a user whose head is on the pillow and a control module 350 that quantitatively operates a sleeping status by detecting parameters required for converting and analyzing a bio-signal detected by the sensor unit 3000 into a digital signal.

Here, the sensor unit 300 includes a vibration/pressure detecting sensor 301 for detecting the variations of the vibration and pressure incurred by the pulse, breathing, body movement, snore, bruxism, upper and lower pads 101 and 12 defining a housing for protecting and fixing the vibration/pressure sensor 301, a fixing support 110 for fixing the upper and lower pads 101 and 102, and an elastic member 305 that is disposed between the sensor and the upper and lower pads to efficiently transfer the vibration and pressure variations to the upper pad.

The control module 350 includes an analog processing unit 411 for extracting necessary information from the output of the sensor unit 300, a control unit 430 operating quantitative values for a pulse rate, a breathing rate, a degree of the body movement, a snore state, and a bruxism state by converting an analog signal into a digital signal, a display unit 509 displaying an analyzing result, and an interface unit 450 for the wire/wireless data communication with an external device 470.

The following will describe operation of the above-described pillow with the unrestricted sleeping status determining apparatus.

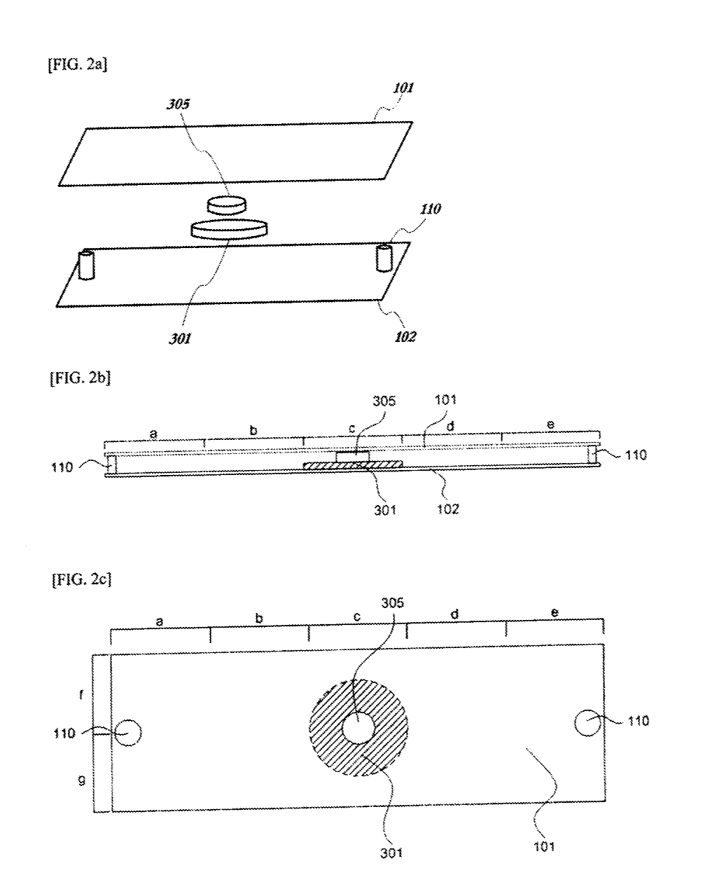

FIGS. 2A to 2C illustrate the sensor unit of the unrestricted sleeping status determining apparatus. That is, FIG. 2A is an exploded perspective view of the sensor unit 300 of the unrestricted sleeping status determining apparatus and FIGS. 2B and 2C are front and top cross-sectional views of the sensor unit 300.

As shown in FIGS. 2A to 2C, the vibration/pressure detecting sensor 301 is installed in the housing defined by the upper and lower pads 101 and 102. When the head of the user is located on the pillow, the vibration/pressure detecting sensor 301 detects information on the movement of the head, which is incurred when the user's body moves, by detecting minute movement of a rear portion of the heat by the heartbeat and breathing and minute vibration of a skull, which is incurred when the user snores or gnashes his/her teeth.

A piezoelectric film or a piezoelectric cable, which coverts mechanical force into electrical force, may be used as the vibration/pressure detecting sensor 301. In order to use the sensor in the pillow, the sensor should be structured to effectively transfer the vibration signal.

That is, since the pillow is generally formed of a flexible material, the vibration and movement of the head may be absorbed by the pillow material and thus the vibration/pressure detecting sensor may not detect the vibration and movement.

Accordingly, a pad 100 in which the vibration/pressure detecting sensor 301 is inserted is first provided. That is, the vibration/pressure detecting sensor 302 is disposed in the pillow in a state where it is inserted in the pad 100.

The housing is defined by the upper and lower pads 101 and 12 that are fixed by the fixing support 110. The vibration/pressure detecting sensor 301 includes the elastic member 305 to transfer the vibration and pressure variations applied to the pad, the elastic member 305.

At this point, as shown in FIGS. 2B and 2C, the vibration/pressure detecting sensor 301 may be located at a center of the pads 101 and 102. When the head of the user is located on a center “c” of the pillow, the vibration and pressure that vary at right above the vibration/pressure detecting sensor 301. Even when the head of the user is located at upper or lower portion “a,” “b,” “d,” “e,” “f,” or “g,” the vibration and pressure variations applied to the pad 101 is transferred to the elastic member 305 of the vibration/pressure detecting sensor 301 located at the center of the pad.

In the sensor unit 300, the vibration/pressure detecting sensor 301 detects the vibration and pressure variations of the user who is sleeping. The detected signal are classified into pulse, breathing, body movement, and snore components by the analog processing unit and analyzed to be used for the inference of the bio-information variation of the user 250 who is sleeping.

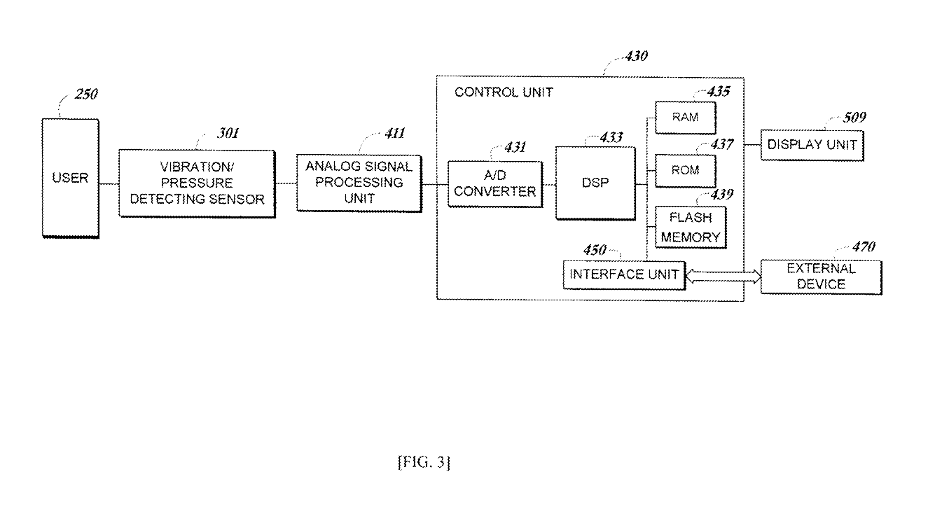

FIG. 3 is a block diagram of the unrestricted sleeping status determining apparatus.

Referring to FIG. 3, in order to detect a variety of bio-information from the user 250 who is sleeping, the signals measured by the sensor unit 300 inserted in the pad 100 and located in the pillow 200 are classified by frequency components of the pulse, breathing, body movement, bruxism, and snore signals and converted into the digital signals by an analog/digital converter 431 of the control unit 430. Then, a DSP 433 performs the sleeping status inferring algorithm operation process for the digital signals and outputs the parameters for estimating the sleeping status.

The quantitative indicator for the sleeping status of the user is attained from the parameters output from the DSP 433. The quantitative indicator is stored in a flash memory 439 of a main control unit. After the user wakes up, the quantitative indicator is displayed on the display unit 509 and transferred to the external device through the wire/wireless interface 450.

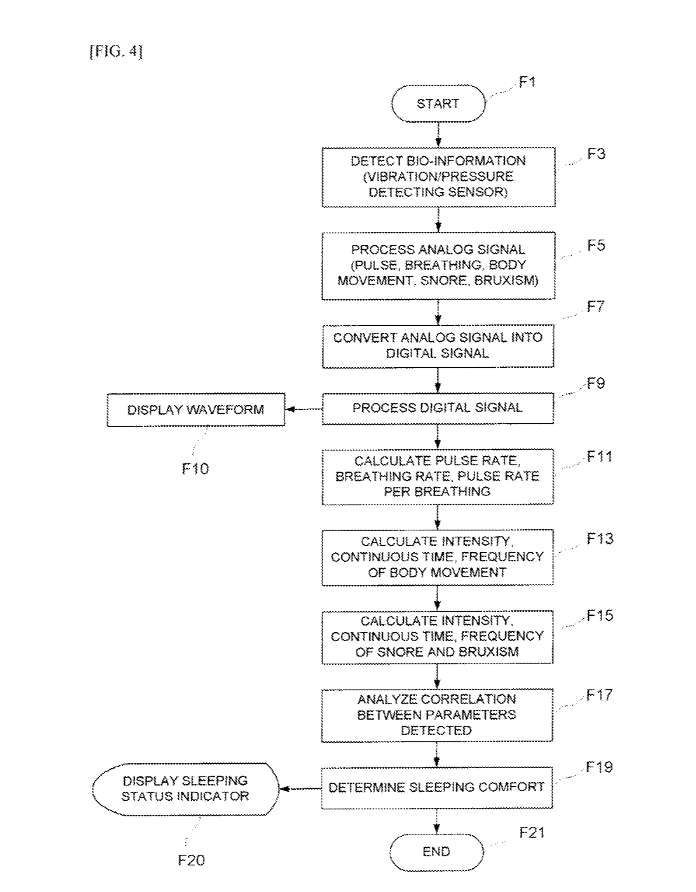

FIG. 4 is a flowchart illustrating the operation of the control unit of the sleeping status determining apparatus.

When the measurement of the variety of the bio-information of the user 250 starts (F1), the bio-signals are output from the vibration/pressure detecting sensor 301 (F3).

The bio-signals are classified by the frequency components of the pulse, breathing, body movement, bruxism, and snore signals through the analog signal processing unit 411 (F5).

The pulse, breathing, body movement, bruxism, and snore signals classified by the analog signal processing unit 411 are converted into the digital signals through the analog/digital converter 431 of the control unit 430 (F7).

The bio-signals converted into the digital signals through the analog/digital converter 431 are classified into pressure variation components of the pulse, breathing, and body movement and vibration variation components of the bruxism and snore (F9) and displayed on the display unit 509 (F10).

Meanwhile, the DSP 433 separates the pulse and breathing waves from the signals that are digitally filtered and calculates pulse rate, breathing rate, and pulse rate per breathing by detecting a peak point of the pulse and starting points of intake and exhaust breathings (F11).

An intensity, continuous time, and frequency for the body movement of the user are calculated by analyzing the body movement signals according to the pressure variation by the body movement (F13). For the sore and bruxism signals. the rectifying and enveloping detection processes are performed. An intensity, continuous time, and frequency of the snore and bruxism are calculated by detecting a portion where a signal above a threshold voltage is generated (F15).

Correlation of the pulse rate and breathing rate and the intensity, continuous time, and frequency for the body movement, snore, and bruxism are analyzed (F17) and the sleeping status are quantitatively determined (F19). The sleeping status indicator is displayed on the display unit 509 (F20).

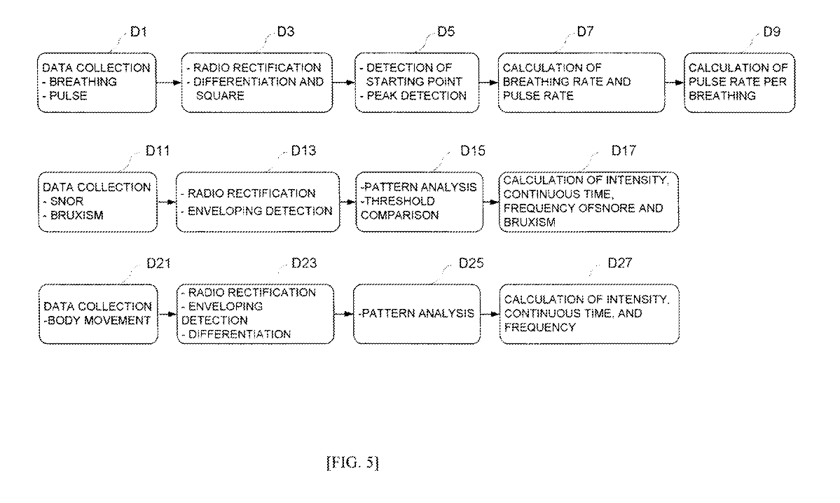

FIG. 5 is a flowchart illustrating a process for deducing parameters from the sleeping status determining apparatus according to one embodiment.

The parameters are deduced by applying a variety of methods to the pulse, breathing, body movement, and snore signals measured by the sleeping status determining apparatus of the embodiment.

The pulse and breathing signals (D1) measured by the apparatus are digitally processed and go through radio rectifying, differential, waveform-integration processes (D3). A starting point and peak of each signal are detected (D5). Using the starting point and peak, the breathing rate and pulse rate for a predetermined time are calculated (D7). The pulse rate per breathing is calculated using a ratio between the breathing rate and the pulse rate. The pulse rate per breathing is used as a useful parameter.

In addition, the snore and bruxism signals (D11) measured by the apparatus go through radio rectifying and enveloping detecting processes (D13). Patterns of the values above the threshold voltage are compared and analyzed (D16) and the intensity, continuous time, and frequency for the sleeping time are calculated (D17). These are used as parameters for determining the sleeping comfort.

The body movement signal (D21) measured by the apparatus goes through the radio rectifying and developing detection and differential processes (D23). The patterns of these waveforms are analyzed (D25). The intensity, continuous time, and frequency of the body movement for the sleeping time are calculated (D27) to determine the body movement degree of the user. These parameters are used for determining the sleeping comfort.

According to the exemplary embodiment, since the sensor for detecting the vibration/pressure variation is inserted in the pad and the pad is installed in the pillow, the bio-information can be attained in an unrestricted state. That is, the measurement for the testee is realized without restriction and self-awareness for the testee and thus the substantial application can be easily done without disturbing the sleep of the testee.

Particularly, since the sleeping status is quantitatively provided by deducing the pulse, breathing, body movement, and snore states from the bio-information that is attained in the unrestricted state, the life pattern can be adjusted in response to the sleeping status and the after-wakeup status.

The pillow with the unrestricted sleeping status determining apparatus of the embodiment is configured to infer the ordinal sleeping status to improve the tiredness, deterioration of the concentration, and the like that may be caused by sleep disorder that may cause the high blood pressure, diabetes, cerebral paralysis, and the like. Based on the inference, the sleeping environment can be improved. The improvement of the sleeping environment results in the improvement of the productivity and reduction of the medical expenses.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A pillow having an apparatus for determining a sleeping state under an unrestricted non-self-awareness condition, the apparatus comprising:
a sensor for detecting analog bio-information on a pulse, breathing, body movement, snore, bruxism for a user putting his/her head on the pillow;
a signal processing unit for filtering off and amplifying the analog bio-information detected by the sensor; and
a control unit for converting the analog bio-information output from the signal processing unit into a digital signal, storing the digital signal therein, extracting parameters from the detected bio-information to provide a sleeping status as a quantitative indicator,
wherein the sensor is installed in the pillow;
the sensor is inserted into a pad type housing so that the sensor does not directly contact an interior material of the pillow; and
the housing is provided on an upper portion of the pillow so that minute vibration and movement of the user's head, which are incurred by the user's breathing, pulse, body movement, and snore, is effectively transferred to the sensor.

2. The pillow of claim 1, wherein the sensor is fixed on a central portion of the pad type housing and is selected from the group consisting of a piezoelectric ceramic, a piezoelectric film, a piezoelectric cable, a resistive pressure sensor, a load cell, and an acceleration sensor.

3. The pillow of claim 1, wherein the housing comprises upper and lower pads for protecting the sensor and a fixing support for fixing the upper and lower pads, wherein the housing is formed of aluminum or plastic so that minute vibration and pressure variation signals during the user's sleep is efficiently transferred to the sensor and the sensor is fixed at the central portion of the housing.

4. The pillow of claim 3, wherein an elastic member is provided between the sensor and the upper pad so that minute vibration and pressure variations are efficiently transferred from the upper pad to the sensor, wherein the elastic member is formed of rubber.
